SEQUENCE LISTING (54 sequences)

SEQ ID NO: 1 - DNA, 16 bp, Homo sapiens
cacgttatcctcctgg

SEQ ID NO: 2 - DNA, 16 bp, Homo sapiens
cacgttatcctcctgg

SEQ ID NO: 3 - DNA, 17 bp, Homo sapiens
cgcacgttatcctcctg

SEQ ID NO: 4 - DNA, 16 bp, Homo sapiens
cgcacgttatcctcct

SEQ ID NO: 5 - DNA, 27 bp, Homo sapiens
ccggatccttcgtgtccccacagcacg

SEQ ID NO: 6 - DNA, 28 bp, Homo sapiens
ccgaattccgctgcactgtgaagctctc

SEQ ID NO: 7 - DNA, 13 bp, Homo sapiens
gactggtgctgct

SEQ ID NO: 8 - DNA, 13 bp, Homo sapiens
gactgatgctgct

SEQ ID NO: 9 - DNA, 13 bp, Homo sapiens
actctaatcacgc

SEQ ID NO: 10 - DNA, 13 bp, Homo sapiens
actctaaccacgc

SEQ ID NO: 11 - DNA, 13 bp, Homo sapiens
actctaaccacgc

SEQ ID NO: 12 - DNA, 13 bp, Homo sapiens
actctaaccacgc

SEQ ID NO: 13 - DNA, 13 bp, Homo sapiens
actctaaccacgc

SEQ ID NO: 14 - DNA, 14 bp, Homo sapiens
ctctaaacacgcgt

SEQ ID NO: 15 - DNA, 14 bp, Homo sapiens
ctctaaccacgcgt

SEQ ID NO: 16 - DNA, 15 bp, Homo sapiens
tgtccaggttccgca

SEQ ID NO: 17 - DNA, 15 bp, Homo sapiens
tgtccaggttccgca

SEQ ID NO: 18 - DNA, 15 bp, Homo sapiens
tgtccaggttccgca

SEQ ID NO: 19 - DNA, 15 bp, Homo sapiens
tgtccaggttccgca

SEQ ID NO: 20 - DNA, 16 bp, Homo sapiens
cacgttatcctcctgg

SEQ ID NO: 21 - DNA, 15 bp, Homo sapiens
tgtccaggttccgca

SEQ ID NO: 22 - DNA, 19 bp, Homo sapiens
tttgatacgacgatagcga

SEQ ID NO: 23 - DNA, 54 bp, Homo sapiens
atgatcagcagcagcagtcaggacgacgacacgcgtgtttagagtagtagatag

SEQ ID NO: 24 - DNA, 54 bp, Homo sapiens
atgatcagcagcaccagtcaggacgacgacacgcgtgattagagtagtagatag

SEQ ID NO: 25 - DNA, 54 bp, Homo sapiens
atgatcagcagcatcagtcaggacgacgacacgcgtgattagagtagtagatag

SEQ ID NO: 26 - DNA, 54 bp, Homo sapiens
atgatcagcagcaccagtcaggatgacgacacgcgtggttagagtagtagatag

SEQ ID NO: 27 - DNA, 54 bp, Homo sapiens
atgatcagcagcatcagtcaggacgacgacacgcgtggttagagtagtagatag

SEQ ID NO: 28 - DNA, 13 bp, Homo sapiens
gactggtgctgct

SEQ ID NO: 29 - DNA, 13 bp, Homo sapiens
gactggtgctgct

SEQ ID NO: 30 - DNA, 25 bp, Homo sapiens
gtcttctagaacgactggtgctgct

SEQ ID NO: 31 - DNA, 13 bp, Homo sapiens
gactggtgctgct

SEQ ID NO: 32 - DNA, 13 bp, Homo sapiens
gactcgagctgct

SEQ ID NO: 33 - DNA, 51 bp, Homo sapiens
gtgcggttgctggaaagatgcatctataaccaagaggagtccgtgcgcttc

SEQ ID NO: 34 - DNA, 51 bp, Homo sapiens
gtgcggtacctggacagatacttccataaccaggaggagaacgtgcgcttc

SEQ ID NO: 35 - DNA, 51 bp, Homo sapiens
gtgcggttcctggagagatacttccataaccaggaggagaacgtgcgcttc

SEQ ID NO: 36 - DNA, 51 bp, Homo sapiens
gtgcggttcctggacagatacttccataaccaggaggagaacgtgcgcttc

SEQ ID NO: 37 - DNA, 51 bp, Homo sapiens; identical to SEQ ID NO: 36

SEQ ID NO: 38 - DNA, 51 bp, Homo sapiens
gtgcggtacctggacagatacttccataaccaggaggagttcctgcgcttc

SEQ ID NO: 39 - DNA, 51 bp, Homo sapiens
gtgcggttcctggagagacacttccataaccaggaggagtacgcgcgcttc

SEQ ID NO: 40 - DNA, 51 bp, Homo sapiens
gtgcggttcctggagagatacttccataaccaggaggagttcgtgcgcttc

SEQ ID NO: 41 - DNA, 51 bp, Homo sapiens
gtgcggttcctggacagatacttctatcaccaagaggagtacgtgcgcttc

SEQ ID NO: 42 - DNA, 51 bp, Homo sapiens
gtgcagttcctggaaagactcttctataaccaggaggagttcgtgcgcttc

SEQ ID NO: 43 - DNA, 51 bp, Homo sapiens
gtgcggttcctggacagatacttctataaccaagaggagtacgtgcgcttc

SEQ ID NO: 44 - DNA, 51 bp, Homo sapiens
gtgcggtatctgcacagaggcatctataaccaagaggagaacgtgcgcttc

SEQ ID NO: 45 - DNA, 51 bp, Homo sapiens
gtgcggttgctggaaagacgcgtccataaccaagaggagtacgcgcgctac

SEQ ID NO: 46 - DNA, 51 bp, Homo sapiens; identical to SEQ ID NO: 43

SEQ ID NO: 47 - DNA, 51 bp, Homo sapiens
gtgcggttactggagagacacttccataaccaggaggagctcctgcgcttc

SEQ ID NO: 48 - DNA, 51 bp, Homo sapiens
gtgcggttcctggacagatacttccataaccaggaggagttcgtgcgcttc

SEQ ID NO: 49 - DNA, 51 bp, Homo sapiens; identical to SEQ ID NO: 35

SEQ ID NO: 50 - DNA, 51 bp, Homo sapiens
gtgcggttcctggacagatacttctataaccaggaggagtccgtgcgcttc

SEQ ID NO: 51 - DNA, 51 bp, Homo sapiens; identical to SEQ ID NO: 50

SEQ ID NO: 52 - DNA, 51 bp, Homo sapiens; identical to SEQ ID NO: 33

SEQ ID NO: 53 - DNA, 51 bp, Homo sapiens
gtgtggaacctgatcagatacatctataaccaagaggagtacgcgcgctac

SEQ ID NO: 54 - DNA, 51 bp, Homo sapiens
gtgcggttcctgcacagagacatctataaccaagaggaggacttgcgcttc

(19) United States
(12) Patent Application Publication
(10) Pub. No.: US 2010/0317004 A1
(43) Pub. Date: Dec. 16, 2010

(54) PROBES

(75) Inventors: Michael BUNCE, Flintshire (GB); Ian Crosby, Wirral (GB)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/717,911

(22) Filed: Mar. 4, 2010

(30) Foreign Application Priority Data
Nov. 22, 2002 (GB) 0227274.8

(51) Int. Cl. C12Q 1/68; C07H 21/04; C12M 1/34

(52) U.S. Cl. 435/6; 536/24.3; 435/287.2

(57) ABSTRACT

The present invention relates to probes, methods and apparatus for the detection of the presence or absence of non-contiguous cis-located nucleic acid sequences which are characteristic of alleles including those relating to the human leukocyte antigen (HLA) which is of interest in the field of human transplantation and disease.

The present invention relates to probes, methods of detection and diagnosis, and apparatus for use in such methods. In particular, the present invention relates to nucleic acid, especially oligonucleotide, probes and to methods and apparatus for the detection of the presence or absence of non-contiguous cis-located nucleic acid sequences and polymorphisms therein.

Two or more nucleic acid polymorphisms on a single molecule or strand are said to be cis-located and these occur naturally in nucleic acid sequences, particularly in alleles belonging to the human leucocyte antigen (HLA) system. As detailed by Bodmer et al (Tissue Antigens (1999) 53 407-446), the HLA locus is the most polymorphic in the human genome. There are currently over 240 HLA-A alleles, 478 HLA-B, 116 HLA-C, 99 HLA-DPB1, 52 DQB1 and 305 DRB1 alleles, with new alleles being discovered continuously. All HLA-A, -B, and -C alleles have similar sequences made up of blocks of polymorphisms, or sequence motifs. The differing alleles are generally made up of recombinations of these sequence motifs. The same holds for class II loci such as DRB1, DRB3, DRB4, DRB5 and DPB1.

Many methods exist for HLA genotyping. One common method utilises target DNA amplification by polymerase chain-reaction (PCR) followed by detection of polymorphic DNA sequences using sequence-specific oligonucleotide probes (SSO) (Saiki R K et al, Nature. 1986 Nov. 13-19; 324(6093):163-6). The SSO probes hybridised to target DNA may be detected by, for example, colorimetric, radioactive or fluorescent methods. In HLA genotyping the initial amplification is normally generic, but may comprise a mosaic of amplifications which when used together amplify all possible alleles of a given locus. SSO techniques were first applied to HLA genotyping (HLA-DQA1) by Saiki et al (Saiki R K et al, Nature. 1986 Nov. 13-19; 324(6093):163-6). Subsequently SSO reverse blot techniques (Erlich H et al, Eur J Immunogenet. 1991 February-April; 18(1-2):33-55) were developed. Reverse methods depend on the incorporation of a chemical label such as biotin in the initial generic PCR amplification, usually via labeled amplification primers. In the reverse dot blot, the SSO probes are bound to a solid support membrane leaving the detection end of the probe free to interact with target DNA. On a single membrane many different probes can be bound which would theoretically contain all of the polymorphisms required to genotype an individual at any given locus. When labeled DNA target is applied to the reverse dot blot membrane the DNA will only hybridise to the probes that are matched in DNA sequence. Hybridised biotin-labeled products are detected by the addition of a reporter molecule that induces a colour change in the substrate. Whether a probe will hybridise specifically to a target DNA sequence is dependent upon the amount of probe-target mismatching, mismatch position relative to the probe and probe length and is largely dependent upon the conditions for the hybridisation (such as temperature and salt concentration).

HLA alleles are constructed from a patchwork of polymorphic DNA motifs that are shared by other alleles. It is not possible to discriminate between certain sets of alleles using conventional SSO techniques due to these shared motifs and the fact that most animals are diploid, in that we all have two copies of each locus, i.e. two HLA-A alleles, two HLA-B etc. As a consequence of shared motifs, a single probe is likely to be capable of detecting a number of different alleles that compromise the specificity of the probe by detecting a large number of alleles rather than a small number of alleles or being specific for one allele.

In addition, using conventional SSO techniques utilising a mixture of probes (one for each spatially separated target polymorphism), it is not possible to determine whether the motifs are present or not on the same allele since each of the probes will hybridise to different alleles if the two alleles present in the DNA both contain one or both of the target polymorphisms.

A theoretical possibility to overcome such problems is to design a probe which will hybridise with two or more target polymorphisms but not with their intervening nucleic acid sequence. However, in circumstances where the target DNA motifs are spaced apart, designing a single probe with standard matching nucleotides complementary to the spatially separated target DNA motifs and the intervening sequence between them will impact on the probe's hybridisation temperature increasing it beyond normal design constraints for conventional SSO techniques where a probe to target DNA hybridization temperature are normally within the range 35° C.-65° C.

U.S. Pat. App. No. 20010019825 discloses a method of amplifying DNA for detecting target nucleic acid sequences with diagnostic primers including primer regions and probe regions which are complementary to target and reference regions respectively on a sample nucleic acid strand. Optionally, there is provided a region on the diagnostic primer that is separated by a spacer region of nucleic acid.

U.S. Pat. App. No. 20020042077 discloses partially non-hybridising oligonucleotides that contain two or more hybridising segments, with any two hybridising segments separated by a non-hybridising spacer segment. The art in this application is the design of probes with multiple hybridising regions, but not specifically to detect cis-located polymorphisms for the purpose of increasing a given probe's specificity for an allele or group of alleles sharing the two or more polymorphisms.

Another major problem associated with probes that comprise two hybridising segments and a non-hybridising spacer segment is that the non-hybridising spacer, being composed either of nucleic acid or a compound with similar properties to that of nucleic acid, produces a probe that requires less stringent hybridisation conditions. The reduced stringency is to allow for hybridisation in the presence of mis-matched nucleotides/nucleosides or similar compounds within the spacer segment, which leads to problems with false positives and results that can be hard to interpret.

It is an object of the present invention to alleviate or overcome one or more of the problems associated with the prior art.

In accordance with a first aspect of the present invention there is provided a nucleic acid probe for detecting a target nucleic acid sequence comprising two or more cis-located regions, the probe comprising two or more non-contiguous nucleic acid regions capable of hybridizing with respective cis-located nucleic acid regions on the target sequence, the non-contiguous regions being separated by one or more spacer regions comprising at least predominantly material which will not hybridise with the target sequence in the region thereof between the cis-located regions and having a length selected with regard to the target sequence effectively to maintain correct spatial orientation of the non-contiguous hybridizing regions on the probe with the cis-located regions on the target sequence such that base-base pairwise hybridisation therebetween can be effected in use of the probe.

The invention relates to probes capable of detecting non-contiguous cis-located nucleic acid sequences which are characteristic of certain alleles including those relating to the human leukocyte antigen (HLA), and other genes within the major histocompatibility complex (MHC) which is of interest in the field of human transplantation and disease. However, the probe of the invention is not restricted to be determinative of genes within the MHC but can be applied to any allelic system in which the alleles have two or more cis-located regions to be detected. For example the probe according to the invention may be designed to target other polymorphic genes including, but not restricted to, thiopurine methyl transferases (TPMT), heamochromatosis gene (HFE), tumour necrosis factor (TNF); lymphotoxin (LT), mannose binding lectin (MBL), ABO and other blood grouping systems such as Secretor, Duffy and Rhesus, Factor V Leiden, platelet membrane glycoproteins (GPIIIa/IIb/Ib/IX), human platelet antigens (IPA), CC-chemokine receptor 5 (CCR5), interleukin genes and interleukin receptors, chemokine genes and chemokine receptors, cystic fibrosis genes, KIR genes, cluster differentiation antigens (CD), such as CD1, NOTCH genes, TOLL genes, heat shock proteins, xanthidin oxidase (XO), manganese superoxide dismutase (SOD), paraxonase, N-acetyl transferase (NAT-1 and NAT2), cytochrome P450 CYP2D6 (debrisoquine hydroxylase), multidrug resistance gene 1 (MDR1) and cell adhesion molecules such as MICA, MICB, VCAM, ICAM and PECAM.

The target nucleic acid sequence may comprise one or more alleles of a gene. For example the target sequence may comprise one or more alleles of HLA. Examples of other alleles that could be determined by the probe according to the invention include, but are not limited to, TPMT*3a, TNF alleles, referred to as the allelic types -238G/-238A-308G/-308A-376G/-376A (see Knight et al Nature Genet. 22: 145-150, 1999), HFE C282Y (Feder et al Proc. Nat. Acad. Sci. 95: 1472-1477, 1998).

The spacer region preferably will not hybridise to any substantial degree with nucleic acid sequences in the target molecule or with other nucleic acid sequences in the sample being probed. This effectively means that the hybridisation conditions under which the probe will hybridise with the target nucleic acid sequence is determined substantially by the nucleotide (or nucleoside) content of the non-contiguous regions of the probe.

The probe of the invention yields a significant advantage over prior art bridged probes in that the length of the spacer region is selected to allow pair-wise matching of the non-contiguous regions with cis-located non-contiguous motifs in a target allele(s), thus making the probe specific for two or more regions when present on the same allele.

The spacer region may comprise, for example, abasic phosphoroamidite ribose molecules, nucleic acid molecules at least substantially mismatched with the target sequence in the intervening region between the cis-located regions, or may comprise any other nucleotide analogue that does not hybridise, or only hybridizes weakly, with the intervening target DNA sequence and which maintains pairwise complimentarity between the two or more hybridizing regions. Preferably, the spacer region comprises a sequence of molecules, each of which are the same, or a similar, size to a nucleic acid base. Thus, the spacer preferably comprises one or more molecules that are spatially correct, having the same or similar spatial dimensions as a nucleic acid base, such as a purine or a pyrimidine. These bases are known to those in the art and include, but are not limited to adenosine derivatives, guanosine derivatives, cytosine derivatives, thymidine derivatives, locked nucleic acids (LNA), peptide nucleic acids (PNA), amino nucleic acids (ANA), 2-amino-2′-deoxyadenosine (2-amino-2′-dA), 2′,3′-aideoxyadenosine (2′,3′-ddA), 3′-deoxyadenosine, cordycepin (3′-dA), 7-deaza-2′-deoxyadenosine (7-deaza-2′-dA), 8-bromo-2′-deoxyadenosine (8-Br-2′-dA), N6-methyl-2′-deoxyadenosine (N6-methyl-2′-dA, 2′,3′-dideoxycytidine (2′,3′-ddC), 5-methyl-2′-deoxycytidine (5-Me-2′-dC), 5-bromo-2′-deoxycytidine (5-Br-2′-dC), 5-iodo-2′-deoxycytidine (5-I-2′-dC), 7-deaza-2′-deoxyguanosine (7-deaza-2′-dG), 8-bromo-2′-deoxyguanosine (8-Br-2′-dG), 4-thio-2′-deoxythymidine (4-thio-dT), 5-C3-carboxy-2′-deoxythymidine (5-C3-carboxy-dT), 5-C6-amino-2′-deoxythymidine (5-C6-amino-dT), inverse-2′-deoxythymidine (inverse-dT), 2′-deoxyuridine (2′-dU), 2,6-diaminopurine-2′-deoxyriboside, 2-aminopurine-2′-deoxyriboside, 6-thio-2′-deoxyguanosine, 7-deaza-2′-deoxyadenosine, 7-deaza-2′-deoxyguanosine, 7-deaza-2′-deoxyxanthosine, 8-bromo-2′-deoxyadenosine, 8-bromo-2′-deoxyguanosine, 8-oxo-2′-deoxyadenosine, 8-oxo-2′-deoxyguanosine, etheno-2′-deoxyadenosine, N6-methyl-2′-deoxyadenosine, O6-methyl-2′-deoxyguanosine, O6-phenyl-2′-deoxyinosine, 2′-deoxypseudouridine, 2′-deoxyuridine, 2-thiothymidine, 4-thio-2′-deoxyuridine, 4-thiothymidine, 4-triazolyl-2′-deoxyuridine, 4-triazolylthymidine, 5′-aminothymidine, 5′-iodothymidine, 5′-O-methylthymidine, 5,6-dihydro-2′-deoxyuridine, 5,6-dihydrothymidine, 5-bromo-2′-deoxycytidine, 5-bromo-2′-deoxyuridine, 5-fluoro-2′-deoxyuridine, 5-hydroxy-2′-deoxyuridine, 5-hydroxymethyl-2′-deoxyuridine, 5-iodo-2′-deoxycytidine, 5-iodo-2′-deoxyuridine, 5-methyl-2′-deoxycytidine, 5-propynyl-2′-deoxycytidine, 5-propynyl-2′-deoxyuridine, carboxy thymidine, N4-ethyl-2′-deoxycytidine, O4-methylthymidine, TMP-F-2′-deoxyuridine, 1-methyladenine, 2-methyladenine, N.sup.6-methyladenine, N.sup.6-isopentyl-adenine, 2-methylthio-N.sup.6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, 5-(2bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine, 3′-thioether-2′,3′-dideoxynucleoside-5′-triphosphates, 3′-thioamido-2′,3′-dideoxynucleoside-5′-triphosphates 3′-allyl-2′,3′-dideoxynucleoside-5′-triphosphates, 3′-urea-2′,3′-dideoxynucleoside-5′-triphosphates, and 3′-thiourea-modified 2′,3′dideoxynucleoside-5′-triphosphates and mismatched purines or pyrimidines.

The spacer region may be interrupted with one or more specific nucleic acids selected to pair with complementary bases on the target nucleic acid sequence, allowing the probe to distinguish mutations or discrete sequences in the region of the target nucleic acid sequence corresponding to the spacer region of the probe on hybridisation. Such a probe may afford greater specificity, particularly where the nucleotide on the target sequence which corresponds to the at least one complementary base is a polymorphism.

The hybridizing regions of the probe comprise nucleic acid regions capable of hybridizing with respective cis-located nucleic acid regions on the target sequence. These nucleic acid regions of the probe will often suitably comprise nucleotides but may alternatively or also comprise modified bases (DNA analogues) which may in some cases bind to the target sequence more efficiently than conventional nucleotides. Suitable DNA analogues include, for example, amino nucleic acid (ANA), peptide nucleic acid (PNA) and locked nucleic acid (LNA). The hybridizing nucleic acid regions of the probe may also comprise nucleosides. The nucleotides, nucleosides, and/or analogues thereof which make up the hybridizing regions of the probe are preferably selected for the property they will specifically hybridise, to an adequate extent, with the target sequence.

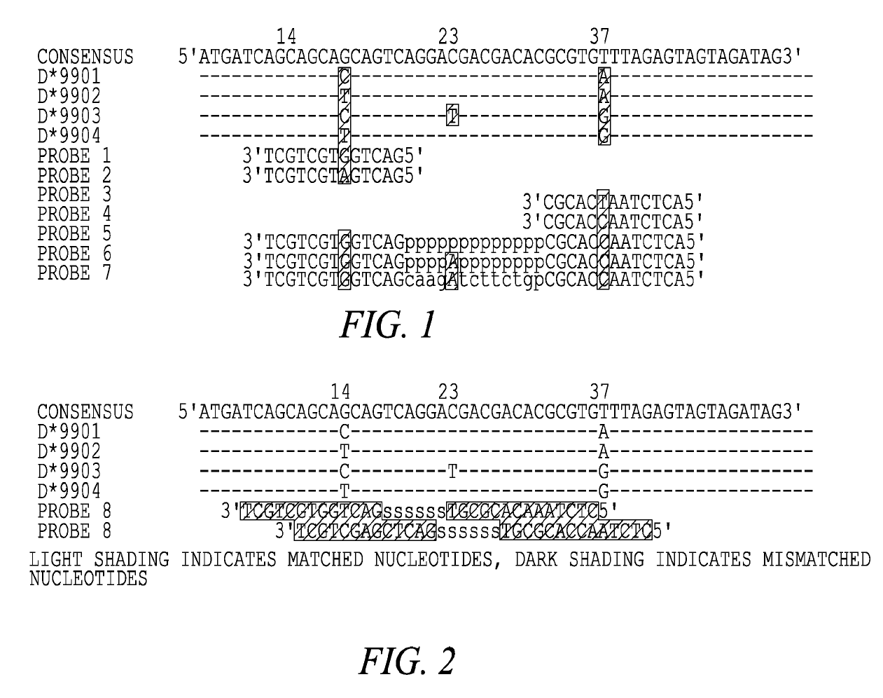

The probe of the invention has a particular advantage over prior art probes in the event that there are a number of polymorphic sequences, one or more of which represent the target sequence, in the sample to be probed. A hypothetical illustration of this advantage is illustrated in FIG. 1. Referring to FIG. 1, SSO probes 1-4 (of the prior art type) could be designed to discriminate between the alleles D*9901-*9904 using polymorphisms at positions 14 and 37. If a locus specific amplification was used and tested with the four probes and hybridized to a locus specific PCR-amplified DNA sample one possible result is that all the probes might be positive. If so, it would not be possible to determine which pair of alleles were present in the sample as D*9901+D*9904 is the same as D*9902+D*9903. However, when the two polymorphisms at positions 14 and 37 are linked (in cis) by a single probe (according to the invention), the probe is specific for the two non-contiguous sequence motifs. In FIG. 1 probe 5 (according to the invention) is specific for the D*9903 allele.

The phosphoroamidite bridge in probe 5 (phosphoramidites in bridge indicated by p) is required to maintain pairwise binding between the probe and the target DNA sequence. Phosphoroamidites are unable to pair-base with target DNA strands and thus do not influence hybridisation temperature. If a probe (of the prior art type) were designed with standard matching nucleotides the probe's hybridisation temperature would be increased above the normal design constraints within an SSO typing system and the probe specificity would be compromised.

Still referring to FIG. 1, probe 6 illustrates a further embodiment of the invention whereby a polymorphism on D*9903 allele is utilised to improve probe specificity by use of a conventional matched nucleotide in the correct spatial orientation in the spacer region of the probe. Probe 7 illustrates a further embodiment of the invention in which the spacer comprises (with one exception corresponding to the aforementioned polymorphism) mismatched nucleotides.

Certain prior art probes are known to contain inert spacers (as described in US-A-2002/0042077, for example). However, the spacers in these prior art probes are not selected (lengthwise) with regard to the target sequence to be effective for maintaining correct spatial orientation, and thus pairwise binding on hybridization, of the non-contiguous regions of the probe with cis-located regions on a target sequence. FIG. 2 illustrates that such prior art probes that contain spacers composed of non-complementary sequences, e.g. incorrect length of spacer, may not provide an adequate or correct pair-wise binding signal due to improper binding. In this example probe 8 has an incorrect spacer length that does not allow correct pairwise binding of the probe hybridization regions to the target nucleic acid regions. In the first instance the left hand arm of the probe hybridizes to the target region but does not allow correct hybridization of the right-hand arm. The situation is reversed in the second example of probe 8 binding. Partial hybridization of probe to incorrect regions of DNA will result in both false positive and false negative results. These examples iterate the problems associated with prior art bridged probes.

The increased specificity owing to the ability of the probe of the invention to hybridise two or more non-contiguous regions of the target sequence reduces the number of alleles which can be detected by that probe and thus provides improved resolution over conventional SSO probing or any other technique involving oligonucleotide probes hybridizing to polymorphic target DNA sequences such as cDNA library screening or detection of RNA polymorphisms. The invention provides for cis (in phase) detection of spatially distinct target nucleic acid sequences with single diagnostic complementary DNA probes. The word cis refers to sequences that are in series on an allele, for example. The phrase ‘spatially distinct’ refers to discrete sequences that are located at different points along a gene or genome.

The probe preferably comprises first and second regions that are capable of hybridising to first and second non-contiguous regions on the target nucleic acid sequence respectively. The regions of the probe that are capable of hybridising to the target nucleic acid sequence may comprise at least one nucleotide complementary to at least one nucleotide on the target nucleic acid sequence. The preferred range of the hybridizing regions is between 1-20 complementary nucleotides.

The probe may comprise the equivalent of 1 to 300 nucleic acid bases in total length. Preferably the probe comprises the equivalent to 1-200 nucleic acid bases. More preferably still, the probe comprises the equivalent to 30-100 nucleic acid bases.

Preferably, the probe is capable of hybridising to HLA alleles, but may include any polymorphic loci where there are two or more polymorphic regions on one or more alleles within said loci.

The probe of the invention may correspond to a formula as follows:

5′-Hyb1-(1-300sp)-Hyb2,
e.g.
5′amino-cacgttatcctcagg(13P)tgtccaggttccgca;
5′amino-cgcacgttatcctcctg(14P)tgtccaggttccgca;
or
5′amino-cgcacgttatcctcct(15P)tgtccaggttccgca,
5′-Hyb1-(1-150sp)-Hyb2-(1-150sp)-Hyb3
e.g.
5′amino-cacgttatcctcctgg(13P)tgtccaggttccgca(17P)
tttgatacgacgatagcga,
5′-Hyb1-[(1-Nsp)-(1-10 nucleotide)-(1-Nsp)-)-
(1-10 nucleotide)-(1-Nsp)]-Hyb2,
e.g.
5′amino-cacgttatcctcctgg(4xP)g(22cP)a(1xP)ta(2xP)
tgtccaggttccgca,

wherein ‘sp’ denotes a spacer molecule, such as a mismatched nucleotide or a phosphoramidite, for example, P=phosphoramidite (by way of an example), Hyb1=first hybridizing nucleotide region comprising, for example, 1-30 nucleotide bases, Hyb2=second hybridizing nucleotide region comprising, for example, 1-30 nucleotide bases and Hyb3=third hybridizing nucleotide region comprising, for example, 1-30 nucleotide bases.

In accordance with a further aspect of the present invention there is provided a method for detecting specifically two or more cis-located target nucleic acid sequence in a sample comprising contacting said sample with at least one probe as described herein above and determining whether any probe/target nucleic acid sequence hybrid is formed.

In accordance with a further aspect of the present invention there is provided a method for detecting specifically non-contiguous cis located polymorphic target nucleic acid sequences in a nucleic acid sample comprising contacting the sample with at least one probe as described hereinabove and determining whether any probe/target nucleic acid sequence hybrid is formed.

Preferably the methods described hereinabove comprise the pre-step of amplifying the nucleic acid sample using a technique known in the art such as, for example, polymerase chain reaction (PCR) (Saiki R K et al Science. 1985 Dec. 20; 230(4732):1350-4).

Preferably, the method of the invention comprises contacting the probes and sample nucleic acid under hybridising conditions. The conditions may be those used in standard SSO techniques.

In accordance with a further aspect of the present invention there is provided an apparatus for detecting a target nucleic acid sequence in a sample, the apparatus comprising a sample application zone and at least one probe as described hereinabove. It is a preferred feature of the present invention that the apparatus for detecting a target nucleic acid sequence in a sample may be used in a biological assay. Preferably, the biological assay is used for the detection of one or more sequences in a sample. More preferably, the biological assay is used for to detect polymorphisms in human leukocyte antigens (HLA).

In accordance with another aspect of the present invention, there is provided a method of diagnosis comprising contacting a probe as herein described, designed to hybridise to an allele or number of alleles and/or mutations, with a sample from an individual and detecting the presence or absence of a resulting hybrid in order to determine the genotype of the individual. This method of diagnosis can be used to assess the precise genetic nature of an individual's condition or disease or to establish the pre-disposition of an individual to a particular condition or disease. Furthermore, the method of diagnosis may also be used to assess genotypic information on an individual. Preferably, the method of diagnosis is used to establish the status of the alleles of the human leukocyte antigens in an individual. It will be apparent to one skilled in the art that the method may be directed towards investigating other polymorphic genes.

In order that the method of the invention may be clearly understood and readily put into effect, a protocol for its operation will now be described. This protocol was used, unless otherwise indicated, in the Examples below.

The probes are conjugated to latex beads via a 5′-amino linker that binds the probe to carboxylate moieties on the bead surface. Briefly, the conjugation process requires incubation of carboxylate beads with amino-labelled oligonucleotides in the presence of 2M 1-Ethyl-3-(3-dimethylaminopropyl)carbodiimide hydrochloride (EDC), and 50 mM N-hydroxysulfosuccinimide (NHS). Un-conjugated probe is subsequently washed away and the beads are resuspended in water and dotted onto nylon membrane (Cuno 0.8 micron) where they are allowed to dry onto the membrane.

Prior to the hybridization assay the sample DNA is PCR amplified: The DNA-containing specimen and reagent mixture are heated to 95° C. to separate the double-stranded DNA and expose the target sequences to the primers. As the mixture cools, the biotinylated primers anneal to their targets. The thermostable recombinant Thermus aquaticus (Taq) DNA polymerase in the presence of excess deoxynucleoside triphosphates (dNTPs), including deoxyadenosine, deoxyguanosine, deoxycytidine and deoxythymidine or deoxyuridine (deoxyuridine for RNA), extends the annealed primers along the target templates to produce a biotinylated DNA sequence termed an amplicon. This process is repeated for a number of cycles, each cycle effectively doubling the amount of target DNA. For this test, an adequate number of cycles has been determined to be 35, theoretically yielding more than a billion-fold amplification.

After the PCR amplification process, the amplicons are chemically denatured (using a solution containing 3% EDTA, 1.6% sodium hydroxide) to form single strands which are then added to a well of a typing tray that contains the nylon membrane with the immobilized, sequence-specific, oligonucleotide probes. The biotin-labeled amplicons then bind (hybridise) to the sequence-specific probes and thus are “captured” onto the membrane strip. The stringent conditions for hybridisation of the amplicons to the probes ensure the specificity of the reaction.

After a stringent wash of the membrane strip to remove unbound material, a streptavidin-horseradish peroxidase (SA-HRP) conjugate is added to the well of the tray. The streptavidin binds to the biotin-labeled amplicons captured by the membrane-bound probe. After washing off unbound conjugate, the bound SA-HRP conjugate is reacted with hydrogen peroxide (H₂O₂) and tetramethylbenzidine (TMB) to form a colour complex. The reaction is stopped by several water washes. The hybridization stages may be performed manually or may be performed automatically using the Dynal AutoRELI™ 48 automated strip development apparatus (available from Dynal Biotech Ltd).

The developed strips can then be scanned using a flat-bed optical scanner and the results can be interpreted manually or by using an analysis program such as Dynal's Pattern Matching Program (PMP).

The present invention will now be described, by way of example only, with reference to the following Figures and Examples, in which:

FIG. 1 illustrates the principle of bridged probes, discussed above;

FIG. 2 illustrates the problem associated with probes that do not contain a correct length bridging sequence to maintain pair-wise binding of hybridizing regions, discussed above.

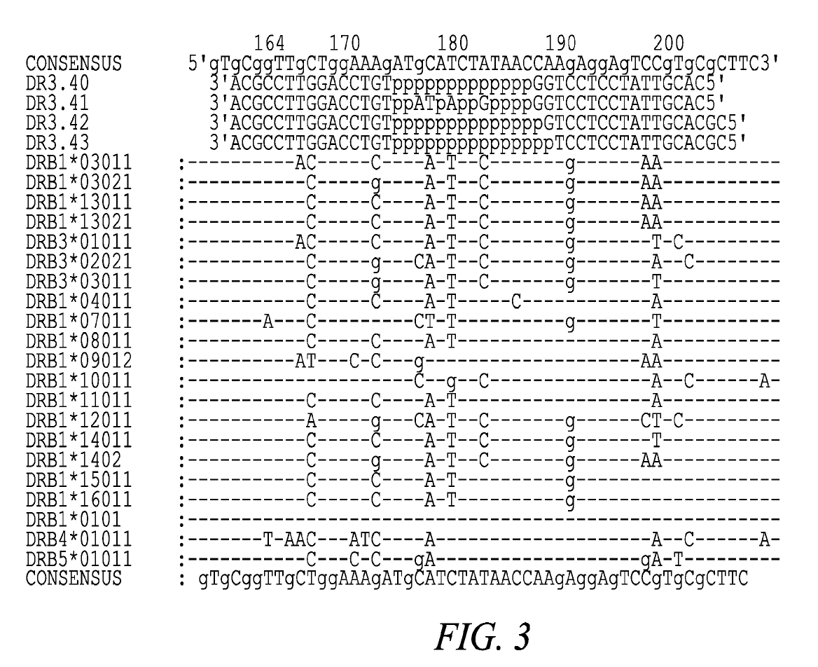

FIG. 3 illustrates probes according to the invention aligned with selected target sequences; and

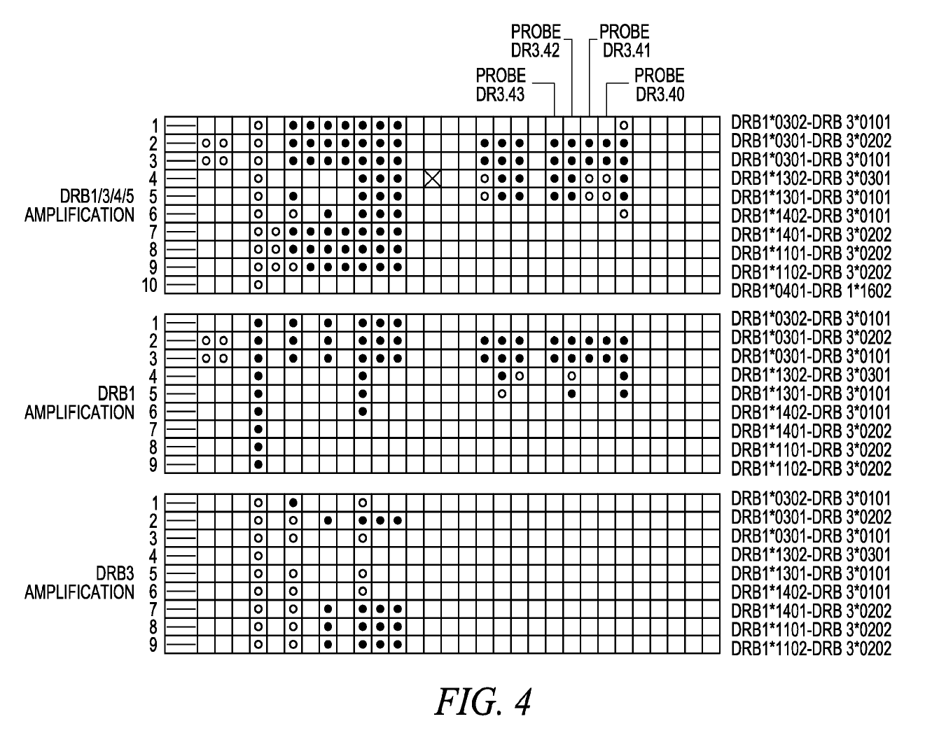

FIG. 4 illustrates the results of probes according to the invention hybridised with nucleic acid samples;

### EXAMPLES

These experiments were conducted in order to investigate the hybridisation of probes specific for certain alleles (DRB1*0301/5 etc). Referring now to FIG. 3, various test probes (see below) were designed for a combination of 164A-165C-172C combined with 190G-197A-198A polymorphisms that together are unique for DRB1*0301/5/6/8/10-13/15/15/18-20 alleles when used on target DNA amplified with DRB1 locus specific primers. The probes DR3.40-43 are bridged. Phosphoramidites indicated by ‘p’. The probes were designed to give positive reactions only when both arms of the bridged probes are matched for an individual allele: the so called ‘intersection specificity’. These probes are designed to have an intersection specificity of DRB1*03011/*03012, *03051/03052, *0308/10/11-16/18-20, *1327/41 & DRB3*0108. Cross-reactivity of the left arm might be expected (for example) with DRB3*0101 alleles whilst cross-reactivity of the right arm might be expected with DkB1*1301 or DRB1*1302 alleles. DNA samples containing these potentially false-positive alleles were tested along with true positive DNA samples for DRB1*0301. The sequences of each probe is outlined in the table below.

| Identity | Sequence |
|---|---|
| DR3.40 | 5′amino-cacgttatcctcctgg(13xp)tgtccaggttc |
| | cgca |
| DR3.41 | 5′amino-cacgttatcctcctgg(4xp)g(2xp)a(1xp) |
| | ta(2xp)tgtccaggttccgca |
| DR3.42 | 5′amino-cgcacgttatcctcctg(14xp)tgtccaggtt |
| | ccgca |
| DR3.43 | 5′amino-cgcacgttatcctcct(15xp)tgtccaggttc |
| | cgca |

Probe Specificity:

The probe region focusing on ˜position 164 (left arm) matches the alleles DRB1*03011/2, DRB1*0304, DRB1*03051/2, DRB1*0306, DRB1*0308, DRB1*0309, DRB1*0310, DRB1*0311, DRB1*0312, DRB1*0313, DRB1*0314, DRB1*0315, DRB1*0316, DRB1*0318, DRB1*0319, DRB1*0320, DRB1*1327, DRB1*1341, DRB3*01011, DRB3*01012, DRB3*0101202, DRB3*01013, DRB3*01014, DRB3*0102, DRB3*0104, DRB3*0106, DRB3*0107, DRB3*0108, DRB3*0110

The second region focusing on ˜position 196 (right arm) matches the alleles DRB1*1608, DRB1*03011, DRB1*03012, DRB1*03021, DRB1*03022, DRB1*0303, DRB1*03051, DRB1*03052, DRB1*0306, DRB1*0307, DRB1*0308, DRB1*0310, DRB1*0311, DRB1*0312, DRB1*0313, DRB1*0314, DRB1*0315, DRB1*0316, DRB1*0318, DRB1*0319, DRB1*0320, DRB1*1109, DRB1*1116, DRB1*1120, DRB1*1140, DRB1*13011, DRB1*13012, DRB1*13021, DRB1*13022, DRB1*1305, DRB1*1306, DRB1*1309, DRB1*1310, DRB1*1315, DRB1*1316, DRB1*1318, DRB1*1320, DRB1*1326, DRB1*1327, DRB1*1328, DRB1*1329, DRB1*1331, DRB1*1332, DRB1*1335, DRB1*1336, DRB1*1339, DRB1*1340, DR111*1341, DRB1*1342, DRB1*1343, DRB1*1402, DRB1*1403, DRB1*1406, DRB1*1409, DRB1*1412, DRB1*1413, DRB1*1417, DRB1*1418, DRB1*1419, DRB1*1421, DRB1*1424, DRB1*1427, DRB1*1429, DRB1*1430, DRB1*1433, DRB3*0108

Intersection Specificity:

Combining both halves of the bridged probes gave an overall specificity for the probe of the following alleles. DRB1*03011/*03012,*03051/03052, 0308/10/11-16/18-20, 1327, 1341 & 3*0108.

Probes were conjugated to 0.1 micron Polysciences latex particles and were subsequently dotted onto Cuno 0.8 micron nylon membrane and allowed to air dry at room temperature. DNA samples were amplified by PCR using generic primers for DRB1/3/4/5 (CRX28 5′-biotin-CCGGATCCTTCGTGTCCCCACAGCACG, AB60 5′-biotin-CCGAATTCCGCTGCACTGTGAAGCTCTC) and alternatively by DRB1 (primers D6 plus DAS6 or D6 plus DAS1 depending on cell line genotype) or DRB3 specific primers D9V plus DAS1 or D9V plus DAS6, again depending on the phenotype). Primers D6, D9V, DAS1 and DAS6 anneal to their targets at positions 15-38, 12-31, 257-278 and 257-278 respectively.

DNA from the following samples were amplified

| | Cell line | | |
|---|---|---|---|
| Position on FIG. 4 | identity | DRB1 | DRB3 |
| 1 | 30 | *0302 | *0101 |
| 2 | 7 | *0301 | *0202 |
| 3 | 104 | *0301 | *0101 |
| 4 | 41 | *1302 | *0301 |
| 5 | 37 | *1301 | *0101 |
| 6 | 21 | *1402 | *0101 |
| 7 | 55 | *1401 | *0202 |
| 8 | 16 | *1101 | *0202 |
| 9 | 17 | *1102 | *0202 |
| 10 | 8 | *0401, | |
| | | *1602 | |

Hybridisation was carried out under the standard protocol described above. The results are shown in FIG. 4 and discussed below.

The results show that probe DR3.41 is specific for DRB1*0301 without cross-reactivity to the closely related DRB1*1301, *1302 or DRB3*0101 alleles. The best specificity is seen with DRB1 amplification as amplification with generic primers (DRB1/3/4/5) results in some cross-reactivity with DRB1*13. Probe DR3.41 is an example of a bridged probe with additional nucleoside matches within the probe bridge.

Probe DR3.40 is an example of a probe with the bridge entirely constructed from phosphoramidites, Probe DR3.40 is most similar to probe DR3.41 but does not have the intervening nucleotides in the bridge, rather the bridge is made up entirely of phosphoramidites. This example is also specific for DRB1*0301 with no cross-reactivity with the closely related DRB1*13 alleles or DRB3*0101 alleles when DRB1-specific amplification is used. If generic amplification with DRB1/3/4/5 primers is used there is some residual cross-reactivity observed with DRB1*13 alleles.

1. A nucleic acid probe for detecting a target nucleic acid sequence comprising two or more cis-located nucleic acid regions, the probe comprising two or more non-contiguous nucleic acid regions capable of hybridizing with respective cis-located nucleic acid regions on the target sequence, the non-contiguous regions being separated by one or more spacer regions comprising at least predominantly material which will not hybridise with the target sequence in the region between the cis-located regions and having a length selected with regard to the target sequence effectively to maintain correct spatial orientation of the non-contiguous hybridizing regions on the probe with the cis-located regions on the target sequence such that base-base pairwise hybridisation therebetween can be effected in use of the probe, the probe comprising one or more nucleic acids matched for hybridization with corresponding one or more nucleic acids in the target sequence in the region between the cis-located regions and/or the two or more non-contiguous nucleic acids being separated by two or more spacer regions.

2. A nucleic acid probe according to claim 1 wherein the length of the spacer region is selected to be substantially equal in length to the length of that region of the target sequence which lies between the cis-located regions.

3. A nucleic acid probe according to claim 1 or claim 2 wherein at least one of the one or more spacer regions comprise(s) a basic phosphoroamidite ribose molecules.

4. A nucleic acid probe according to any one of claims 1 to 3 wherein at least one of the one or more spacer regions comprise(s) a nucleic acid sequence mismatched with respect to the target sequence in the region between the cis-located regions.

5. A nucleic acid probe according to any one of claims 1 to 4 wherein the probe comprises the equivalent to 1 to 300 nucleotide bases.

6. A nucleic acid probe according to any one of claims 1 to 5 wherein the target nucleic acid sequence comprises one or more alleles of a gene.

7. A nucleic acid probe according to any one of claims 1 to 6 wherein the target nucleic acid sequence comprises the human leukocyte antigen gene (HLA).

8. A nucleic add probe according to any one of claims 1 to 6 wherein the target nucleic acid sequence comprises the heamochromatosis gene (HFE).

9. A nucleic acid probe according to any one of claims 1 to 7 wherein the target nucleic acid sequence comprises the thiopurine methyl transferase gene (TPMT).

10. A nucleic acid probe according to any one of claims 1 to 6 wherein the target nucleic acid sequence comprises the tumour necrosis factor gene (TNF).

11. A nucleic acid probe according to any one of claims 1 to 10 wherein the probe comprises any one or more sequences selected from the following group:
5′-Hyb1-(1-300sp)-Hyb2,
e.g.
5′amino-cacgttatcctcagg(13P)tgtccaggttccgca;
5′amino-cgcacgttatcctcctg(14P)tgtccaggttccgca;
or
5′amino-cgcacgttatcctcct(15P)tgtccaggttccgca,
5′-Hyb1-(1-150sp)-Hyb2-(1-150sp)-Hyb3
e.g.
5′amino-cacgttatcctcctgg(13P)tgtccaggttccgca(17P)
tttgatacgacgatagcga,
5′-Hyb1-+(1-Nsp)-(1-10 nucleotide)-(1-Nsp)-)-
(1-10 nucleotide)-(1-Nsp)+-Hyb2,
e.g.
5′amino-cacgttatcctcctgg(4xP)g(22cP)a(1xP)ta(2xP)
tgtccaggttccgca,
wherein ‘sp’ denotes a spacer molecule, such as a mismatched nucleotide or a phosphoramidite, for example, P=phosphoramidite (by way of an example), Hyb1=first hybridizing nucleotide region comprising, for example, 1-30 nucleotide bases, Hyb2=second hybridizing nucleotide region comprising, for example, 1-30 nucleotide bases and Hyb3=third hybridizing nucleotide region comprising, for example, 1-30 nucleotide bases.

12. A probe kit comprising first and second nucleic acid probes according to any one of claims 1 to 11, the first probe differing from the second by at least one nucleic acid base in at least one of the non-contiguous regions such that the first probe is capable of hybridizing with a first target sequence and the second probe is capable of hybridizing with a second, polymorphic, target sequence.

13. A method for detecting a target nucleic acid sequence in a sample comprising contacting said sample with at least one nucleic acid probe according to any one of claims 1 to 11 and determining the presence of any hybridized material.

14. A method according to claim 13 comprising a pre-step of amplifying the nucleic acid sample using the polymerase chain reaction (PCR).

15. A method according to claim 13 or claim 15 comprising a step of contacting the probe and sample nucleic acid under hybridising conditions.

16. A method according to any one of claims 13 to 15 comprising a step of contacting the probes and sample nucleic under a standard sequence-specific oligonucleotide (SSO) protocol.

17. An apparatus for detecting a target nucleic acid sequence in a sample, wherein the apparatus comprises a sample application zone and at least one probe according to anyone of claims 1 to 11.

18. An apparatus according to claim 17, wherein the apparatus is used in a biological assay.

19. An apparatus according to claim 18, wherein the biological assay is used for the detection of one or more sequences in a sample.

20. An apparatus according to any one of claims 17 to 19 wherein the biological assay is used to detect polymorphisms in human leukocyte antigens (HLA).

21. A method for detecting a target nucleic acid sequence in a sample comprising contacting a sample with at least one probe according to claims 1 to 11, and determining whether any at least one probe/target nucleic acid sequence hybrid is formed

22. A method of diagnosis comprising contacting a probe according to claims 1 to 11, designed to hybridise to an allele or number of alleles and/or mutations with a sample from an individual in order to determine the genotype of the individual.

23. A method of diagnosis according to claim 22, wherein method of diagnosis is used to establish the status of the alleles of human leukocyte antigens in an individual.

24. A nucleic acid probe for use in a method of detection and an apparatus for detection of a target nucleic acid sequence in a sample thereof substantially as herein described with reference to and as illustrated in the accompanying drawings.
